(19) United States
(12) Patent Application Publication
(10) Pub. No.: US 2025/0262434 A1
(43) Pub. Date: Aug. 21, 2025

(54) COCHLEAR IMPLANT SYSTEM WITH ACOUSTIC ENVIRONMENT MONITORING

(71) Applicant: Envoy Medical Corporation, White Bear Lake, MN (US)

(72) Inventors: Paul R. Mazanec, Ham Lake, MN (US); Brent Thomas Lucas, North Oaks, MN (US); Thomas B. Hoegh, Edina, MN (US)

(21) Appl. No.: 19/054,344

(22) Filed: Feb. 14, 2025

Related U.S. Application Data
(60) Provisional application No. 63/554,777, filed on Feb. 16, 2024.

(51) Int. Cl. A61N 1/36; A61N 1/372; A61N 1/378

(52) U.S. Cl. CPC A61N 1/36038; A61N 1/36125; A61N 1/36128; A61N 1/37217; A61N 1/37247; A61N 1/378

(57) ABSTRACT

A cochlear implant and analysis system includes a stimulator, a first input source, a signal processor, a second input source, and an external device. The system can receive an acoustic stimulus, generate a first input signal from the first input source representative of the acoustic stimulus, and apply a transfer function to the input signal to generate and output a stimulation signal to the stimulator. The second input source can receive the acoustic stimulus and generate a second input signal representative thereof. The external device can receive and store the stimulation signal, the second input signal, and optionally the first input signal in a memory. The external device can also synchronize and compare the stimulation signal, the second input signal, and optionally the first input signal.

### CROSS-REFERENCE

This application claims priority to U.S. Provisional Patent Application 63/554,777, filed Feb. 16, 2024, the entire contents of which is incorporated herein by reference.

### BACKGROUND

A cochlear implant is an electronic device that may be at least partially implanted surgically into the cochlea, the hearing organ of the inner ear, to provide improved hearing to a patient. Cochlear implants may include components that are worn externally by the patient and components that are implanted internally in the patient. Implant system can receive acoustic stimuli and output electrical stimuli to a patient in response thereto to effectively enable a patient to hear. However, a patient with an implant system may have difficulty perceiving the acoustic stimuli correctly due to various factors of the electrical stimuli.

### SUMMARY

Some aspects of the disclosure include cochlear implant and analysis systems. Such systems can include a cochlear electrode, a stimulator in electrical communication with the cochlear electrode, an input source, and a signal processor. The signal processor can be configured to receive an input signal from the input source and output a stimulation signal to the stimulator based on the received input signal and a transfer function of the signal processor.

One embodiment of the present disclosure includes a cochlear implant and analysis system. The cochlear implant and analysis system can comprise an implantable cochlear implant system. The implantable cochlear implant system can include a stimulator, configured to provide electrical stimulation, and a first input source, configured to receive a first input representative of an ambient sound and generate a first input signal representative of the received first input. The implantable cochlear implant system can also include a first signal processor in communication with the stimulator and the first input source. The first signal processor can be programmed such that the first signal processor generates and outputs a stimulation signal to the stimulator based, at least in part, on the first input signal received from the first input source. The cochlear implant and analysis system can also comprise a second input source configured to receive the ambient sound and generate a second input signal representative of the ambient sound. The cochlear implant and analysis system can additionally include an external device in wireless communication with the implantable cochlear implant system and in communication with the second input source. The external device can include a second signal processor. The second signal processor can be programmed to receive the stimulation signal generated by the first signal processor, receive the second input signal from the second input source, and store the stimulation signal and the second input signal in a memory.

One embodiment of the present disclosure includes a method of monitoring signals for a cochlear implant system. The method can include receiving a first input representative of an ambient sound at a first input source and generating a first input signal representative of the received first input. The method can also include generating and outputting, via a first signal processor, a stimulation signal to a stimulator based at least in part on the first input signal received from the first input source and receiving the ambient sound at a second input source. The method can additionally include generating, via the second input source, a second input signal representative of the ambient sound and receiving, at an external device, the stimulation signal generated by the first signal processor and the second input signal generated by the second input source. The method can also include storing the stimulation signal and the second input signal in a memory.

One embodiment of the present disclosure includes a method of adjusting a cochlear implant system. The method can include pairing a cochlear implant system with an external device comprising a speaker. The cochlear implant system can include an input source configured to generate an input signal representative of ambient sound and a signal processor programmed to output a stimulation signal to a stimulator based in part on the input signal. The method can further include receiving a selection of a phoneme, word, or phrase via the external device and outputting the selection of the phoneme, word, or phrase to the cochlear implant system via the speaker. The method can additionally include receiving an indication of an accuracy of the phoneme, word, or phrase via the external device and adjusting at least one of the input source, the signal processor, the stimulator, or a programmed transfer function of the signal processor, based on the received indication of the accuracy of the phoneme, word, or phrase.

### BRIEF DESCRIPTION OF THE DRAWINGS

FIG. 1 shows a schematic illustration of a fully implantable cochlear implant system.

FIG. 2 shows an embodiment of a fully-implantable cochlear implant.

FIG. 3 illustrates an embodiment of an exemplary middle ear sensor for use in conjunction with anatomical features of a patient.

FIG. 4 is a schematic diagram illustrating an exemplary implantable system including an acoustic stimulator.

FIG. 5 is a high lev el electrical schematic showing communication between the implantable battery and/or communication module and the signal processor.

FIG. 6A is a schematic diagram showing an exemplary signal processing configuration for adapting to variability in a sensor frequency response.

FIG. 6B shows an exemplary gain vs. frequency response curve for signals at various stages in the processing configuration.

FIG. 7 is a schematic diagram of an example cochlear implant and analysis system including an exemplary implantable cochlear implant system and an external device.

FIG. 8 is a flow chart diagram of an example method of monitoring signals of an example cochlear implant and analysis system.

FIG. 9 is a flow chart diagram of another example method of monitoring signals of an example cochlear implant and analysis system.

FIG. 10 is a flow chart diagram of an example method of analyzing signals of an example cochlear implant and analysis system.

FIG. 11 is a flow chart diagram of an example method of updating a transfer function based on detected confusion between phonemes.

### DETAILED DESCRIPTION

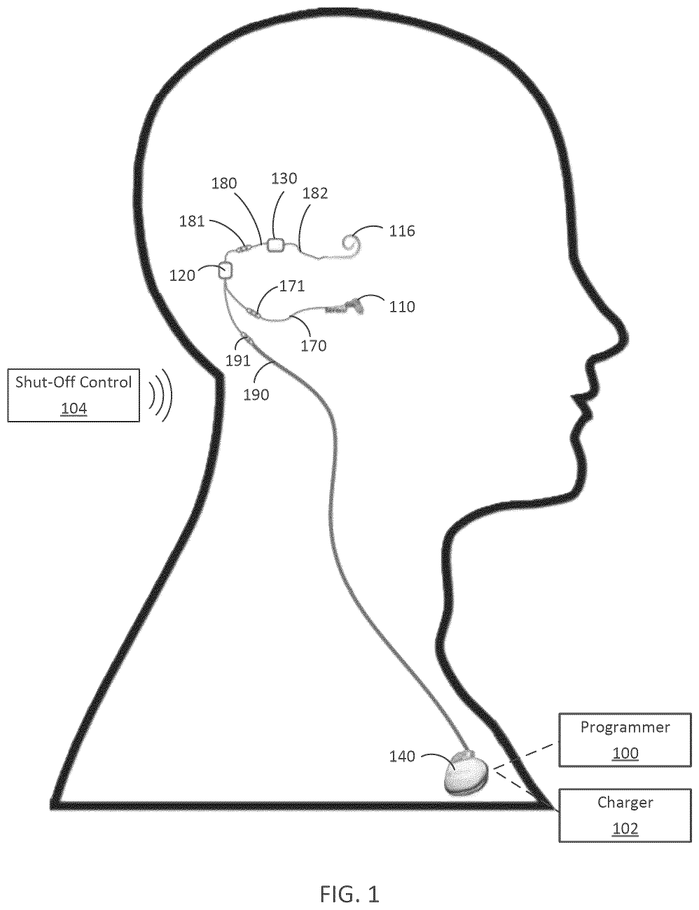

FIG. 1 shows a schematic illustration of a fully implantable cochlear implant system. The system of FIG. 1 includes a middle ear sensor 110 in communication with a signal processor 120. The middle ear sensor 110 can be configured to detect incoming sound waves, for example, using the ear structure of a patient. The signal processor 120 can be configured to receive a signal from the middle ear sensor 110 and produce an output signal based thereon. For example, the signal processor 120 can be programmed with instructions to output a certain signal based on a received signal. In some embodiments, the output of the signal processor 120 can be calculated using an equation based on received input signals. Alternatively, in some embodiments, the output of the signal processor 120 can be based on a lookup table or other programmed (e.g., in memory) correspondence between the input signal from the middle ear sensor 110 and the output signal. While not necessarily based explicitly on a function, the relationship between the input to the signal processor 120 (e.g., from the middle ear sensor 110) and the output of the signal processor 120 is referred to as the transfer function of the signal processor 120.

In various examples, the signal processor 120 can comprise any variety of components, for example, digital and/or analog processing components. In some embodiments, signal processor 120 comprises a digital signal processor, one or more microprocessors, microcontrollers, application specific integrated circuits (ASICs) or the like. Supporting circuitry for one or more such components can also be included as a part of the signal processor. In some embodiments, the signal processor can include or otherwise communicate with a memory containing programming for operating one or more components. Additionally or alternatively, in some embodiments, the signal processor can include one or more additional components. For example, in some embodiments, signal processor can include an embedded microphone or other sensor configured to detect incoming sound waves.

The system of FIG. 1 further includes a cochlear electrode 116 implanted into the cochlear tissues of a patient. The cochlear electrode 116 is in electrical communication with an electrical stimulator 130, which can be configured to provide electrical signals to the cochlear electrode 116 in response to input signals received by the electrical stimulator 130. In some examples, the cochlear electrode 116 is fixedly attached to the electrical stimulator 130. In other examples, the cochlear electrode 116 is removably attached to the electrical stimulator 130. As shown, the electrical stimulator 130 is in communication with the signal processor 120. In some embodiments, the electrical stimulator 130 provides electrical signals to the cochlear electrode 116 based on output signals from the signal processor 120.

In various embodiments, the cochlear electrode 116 can include any number of contact electrodes in electrical contact with different parts of the cochlear tissue. In such embodiments, the electrical stimulator 130 can be configured to provide electrical signals to any number of such contact electrodes to stimulate the cochlear tissue. For example, in some embodiments, the electrical stimulator 130 is configured to activate different contact electrodes or combinations of contact electrodes of the cochlear electrode 116 in response to different input signals received from the signal processor 120. This can help the patient differentiate between different input signals.

During exemplary operation, the middle ear sensor 110 detects audio signals, for example, using features of the patient's ear anatomy as described elsewhere herein and in U.S. Patent Publication No. 2013/0018216, which is hereby incorporated by reference in its entirety. The signal processor 120 can receive such signals from the middle ear sensor 110 and produce an output to the electrical stimulator 130 based on the transfer function of the signal processor 120. The electrical stimulator 130 can then stimulate one or more contact electrodes of the cochlear electrode 116 based on the received signals from the signal processor 120.

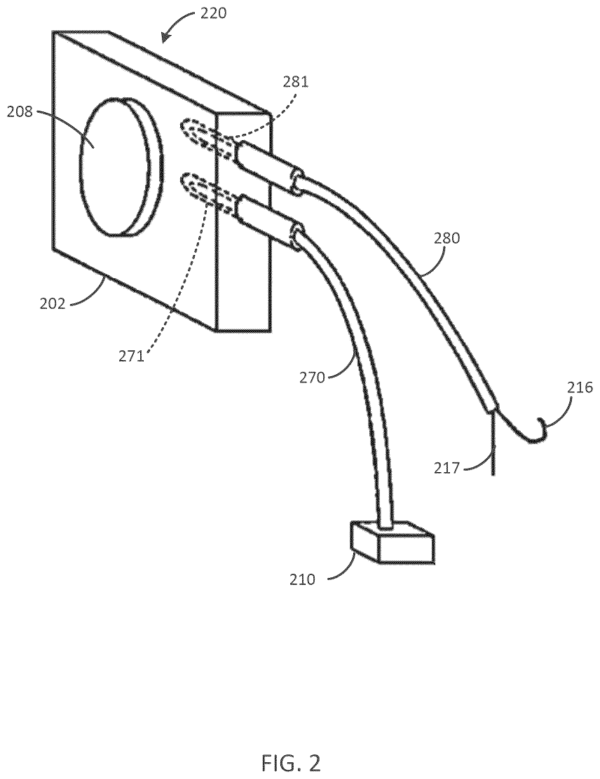

Referring to FIG. 2, an embodiment of a fully-implantable cochlear implant is shown. The device in this embodiment includes a processor 220 (e.g., signal processor), a sensor 210, a first lead 270 connecting the sensor 210 to the processor 220, and a combination lead 280 attached to the processor 220, wherein combination lead 280 contains both a ground electrode 217 and a cochlear electrode 216. The illustrated processor 220 includes a housing 202, a coil 208, first female receptacle 271 and second female receptacle 281 for insertion of the leads 270 and 280, respectively.

In some embodiments, coil 208 can receive power and/or data from an external device, for instance, including a transmission coil (not shown). Some such examples are described in U.S. Patent Publication No. 2013/0018216, which is incorporated by reference. In other examples, processor 220 is configured to receive power and/or data from other sources, such as an implantable battery and/or communication module as shown in FIG. 1. Such battery and/or communication module can be implanted, for example, into the pectoral region of the patient in order to provide adequate room for larger equipment (e.g., a relatively large battery) for prolonged operation (e.g., longer battery life). Additionally, in the event a battery needs eventual replacement, a replacement procedure in the patient's pectoral region can be performed several times without certain vascularization issues that can arise near the location of the cochlear implant. For example, in some cases, repeated procedures (e.g., battery replacement) near the cochlear implant can result in a decreased ability for the skin in the region to heal after a procedure. Placing a replaceable component such as a battery in the pectoral region can facilitate replacement procedures with reduced risk for such issues.

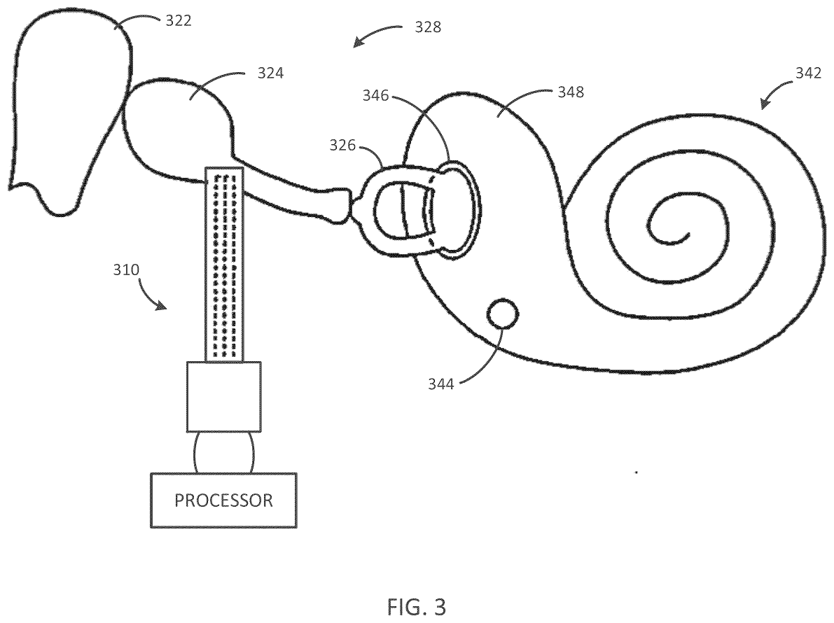

FIG. 3 illustrates embodiments of an exemplary middle ear sensor for use in conjunction with anatomical features of a patient. Referring to FIG. 3, an embodiment of the sensor 310 of a fully-implantable cochlear implant is shown. Also shown are portions of the subject's anatomy, which includes, if the subject is anatomically normal, at least the malleus 322, incus 324, and stapes 326 of the middle ear 328, and the cochlea 348, oval window 346, and round window 344 of the inner ear 342. Here, the sensor 310 is touching the incus 324. The sensor 310 can include a sensor such as described in U.S. Patent Publication No. 2013/0018216, which is incorporated by reference. Further, although not shown in a drawing, the sensor 310 may be in operative contact with the tympanic membrane or the stapes, or any combination of the tympanic membrane, malleus 322, incus 324, or stapes 326.

FIG. 3 illustrates an exemplary middle ear sensor for use with systems described herein. However, other middle ear sensors can be used, such as sensors using microphones or other sensors capable of receiving an input corresponding to detected sound and outputting a corresponding signal to the signal processor. Additionally or alternatively, systems can include other sensors configured to output a signal representative of sound received at or near a user's ear, such as a microphone or other acoustic pickup located in the user's outer ear or implanted under the user's skin. Such devices may function as an input source, for example, to the signal processor such that the signal processor receives an input signal from the input source and generates and output one or more stimulation signals according to the received input signal and the signal processor transfer function. Additionally or alternatively, systems can include other types of sensors, such as inner ear sensors. Some example configurations of such systems and other sensor arrangements are described in PCT patent application No. PCT/US20/19166, filed Feb. 21, 2020, which is assigned to the assignee of the instant application and is incorporated by reference.

Referring back to FIG. 1, the signal processor 120 is shown as being in communication with the middle ear sensor 110, the electrical stimulator 130, and the implantable battery and/or communication module 140. As described elsewhere herein, the signal processor 120 can receive input signals from the middle ear sensor 110 and/or other input source(s) and output signals to the electrical stimulator 130 for stimulating the cochlear electrode 116. The signal processor 120 can receive data (e.g., processing data establishing or updating the transfer function of the signal processor 120) and/or power from the implantable battery and/or communication module 140.

In some embodiments, the implantable battery and/or communication module 140 can communicate with one or more external components, such as a programmer 100 and/or a battery charger 102. The battery charger 102 can wirelessly charge the battery in the implantable battery and/or communication module 140 when brought into proximity with the implantable battery and/or communication module 140 in the pectoral region of the patient. Such charging can be accomplished, for example, using inductive charging. The programmer 100 can be configured to wirelessly communicate with the implantable battery and/or communication module 140 via any appropriate wireless communication technology, such as Bluetooth, Wi-Fi, and the like. In some examples, the programmer 100 can be used to update the system firmware and/or software. In an exemplary operation, the programmer 100 can be used to communicate an updated signal processor 120 transfer function to the implantable battery and/or communication module 140. In various embodiments, the programmer 100 and charger 102 can be separate devices or can be integrated into a single device.

In the illustrated example of FIG. 1, the signal processor 120 is connected to the middle ear sensor 110 via lead 170. In some embodiments, lead 170 can provide communication between the signal processor 120 and the middle ear sensor 110. In some embodiments, lead 170 can include a plurality of isolated conductors providing a plurality of communication channels between the middle ear sensor 110 and the signal processor 120. The lead 170 can include a coating such as an electrically insulating sheath to minimize any conduction of electrical signals to the body of the patient. In various embodiments, one or more communication leads can be detachable such that communication between two components can be disconnected in order to electrically and/or mechanically separate such components. For instance, in some embodiments, lead 170 includes a detachable connector 171. Detachable connector 171 can facilitate decoupling of the signal processor 120 and middle ear sensor 110. Example detachable connectors are described in PCT patent application No. PCT/US20/19166, which is incorporated by reference. For example, with reference to FIG. 1, in some embodiments, lead 170 can include a first lead extending from the middle ear sensor 110 having one of a male or a female connector and a second lead extending from the signal processor 120 having the other of the male or female connector. The first and second leads can be connected at detachable connector 171 in order to facilitate communication between the middle ear sensor 110 and the signal processor 120.

In other examples, a part of the detachable connector 171 can be integrated into one of the middle ear sensor 110 and the signal processor 120. For example, in an exemplary embodiment, the signal processor 120 can include a female connector integrated into a housing of the signal processor 120. Lead 170 can extend fully from the middle ear sensor 110 and terminate at a corresponding male connector for inserting into the female connector of the signal processor 120. In still further embodiments, a lead (e.g., 170) can include connectors on each end configured to detachably connect with connectors integrated into each of the components in communication. For example, lead 170 can include two male connectors, two female connectors, or one male and one female connector for detachably connecting with corresponding connectors integral to the middle ear sensor 110 and the signal processor 120. Thus, lead 170 may include two or more detachable connectors.

Similar communication configurations can be established for detachable connector 181 of lead 180 facilitating communication between the signal processor 120 and the stimulator 130 and for detachable connector 191 of lead 190 facilitating communication between the signal processor 120 and the implantable battery and/or communication module 140. Leads (170, 180, 190) can include pairs of leads having corresponding connectors extending from each piece of communicating equipment, or connectors can be built in to any one or more communicating components.

In such configurations, each of the electrical stimulator 130, signal processor 120, middle ear sensor 110, and battery and/or communication module can each be enclosed in a housing, such as a hermetically sealed housing comprising biocompatible materials. Such components can include feedthroughs providing communication to internal components enclosed in the housing. Feedthroughs can provide electrical communication to the component via leads extending from the housing and/or connectors integrated into the components.

In a module configuration such as that shown in FIG. 1, various components can be accessed (e.g., for upgrades, repair, replacement, etc.) individually from other components. For example, as signal processor 120 technology improves (e.g., improvements in size, processing speed, power consumption, etc.), the signal processor 120 implanted as part of the system can be removed and replaced independently of other components. In an exemplary procedure, an implanted signal processor 120 can be disconnected from the electrical stimulator 130 by disconnecting detachable connector 181, from the middle ear sensor 110 by disconnecting detachable connector 171, and from the implantable battery and/or communication module 140 by disconnecting detachable connector 191. Thus, the signal processor 120 can be removed from the patient while other components such as the electrical stimulator 130, cochlear electrode 116, middle ear sensor 110, and battery and/or communication module can remain in place in the patient.

After the old signal processor is removed, a new signal processor can be connected to the electrical stimulator 130, middle ear sensor 110, and implantable battery and/or communication module 140 via detachable connectors 181, 171, and 191, respectively. Thus, the signal processor (e.g., 120) can be replaced, repaired, upgraded, or any combination thereof, without affecting the other system components. This can reduce, among other things, the risk, complexity, duration, and recovery time of such a procedure. In particular, the cochlear electrode 116 can be left in place in the patient's cochlea while other system components can be adjusted, reducing trauma to the patient's cochlear tissue.

Such modularity of system components can be particularly advantageous when replacing a signal processor 120, such as described above. Processor technology continues to improve and will likely continue to markedly improve in the future, making the signal processor 120 a likely candidate for significant upgrades and/or replacement during the patient's lifetime. Additionally, in embodiments such as the embodiment shown in FIG. 1, the signal processor 120 communicates with many system components. For example, as shown, the signal processor 120 is in communication with each of the electrical stimulator 130, the middle ear sensor 110, and the implantable battery and/or communication module 140. Detachably connecting such components with the signal processor 120 (e.g., via detachable connectors 181, 171, and 191) enables replacement of the signal processor 120 without disturbing any other components. Thus, in the event of an available signal processor 120 upgrade and/or a failure of the signal processor 120, the signal processor 120 can be disconnected from other system components and removed.

While many advantages exist for a replaceable signal processor 120, the modularity of other system components can be similarly advantageous, for example, for upgrading any system component. Similarly, if a system component (e.g., the middle ear sensor 110) should fail, the component can be disconnected from the rest of the system (e.g., via detachable connector 171) and replaced without disturbing the remaining system components. In another example, even a rechargeable battery included in the implantable battery and/or communication module 140 may eventually wear out and need replacement. The implantable battery and/or communication module 140 can be replaced or accessed (e.g., for replacing the battery) without disturbing other system components. Further, as discussed elsewhere herein, when the implantable battery and/or communication module 140 is implanted in the pectoral region of the patient, such as in the illustrated example, such a procedure can leave the patient's head untouched, eliminating unnecessarily frequent access beneath the skin.

While various components are described herein as being detachable, in various embodiments, one or more components configured to communicate with one another can be integrated into a single housing. For example, in some embodiments, signal processor 120 can be integrally formed with the stimulator 130 and cochlear electrode 116. For example, in an exemplary embodiment, processing and stimulation circuitry of a signal processor 120 and stimulator 130 can be integrally formed as a single unit in a housing coupled to a cochlear electrode. Cochlear electrode and the signal processor/stimulator can be implanted during an initial procedure and operate as a single unit.

In some embodiments, while the integral signal processor/stimulator/cochlear electrode component does not get removed from a patient due to potential damage to the cochlear tissue into which the cochlear electrode is implanted, system upgrades are still possible. For example, in some embodiments, a modular signal processor may be implanted alongside the integral signal processor/stimulator component and communicate therewith. In some such examples, the integral signal processor may include a built-in bypass to allow a later-implanted signal processor to interface directly with the stimulator. Additionally or alternatively, the modular signal processor can communicate with the integral signal processor, which may be programmed with a unity transfer function. Thus, in some such embodiments, signals from the modular signal processor may be passed through the integral signal processor unchanged so that the modular signal processor effectively controls action of the integral stimulator. Thus, in various embodiments, hardware and/or software solutions exist for upgrading an integrally attached signal processor that may be difficult or dangerous to remove.

While often described herein as using an electrical stimulator to stimulate the patient's cochlear tissue via a cochlear electrode, in some examples, the system can additionally or alternatively include an acoustic stimulator. An acoustic stimulator can include, for example, a transducer (e.g., a piezoelectric transducer) configured to provide mechanical stimulation to the patient's ear structure. In an exemplary embodiment, the acoustic stimulator can be configured to stimulate one or more portions of the patient's ossicular chain via amplified vibrations. Acoustic stimulators can include any appropriate acoustic stimulators, such as those found in the ESTEEM™ implant (Envoy Medical Corp., St. Paul, Minn.) or as described in U.S. Pat. Nos. 4,729,366, 4,850,962, and 7,524,278, and U.S. Patent Publication No. 20100042183, each of which is incorporated herein by reference in its entirety.

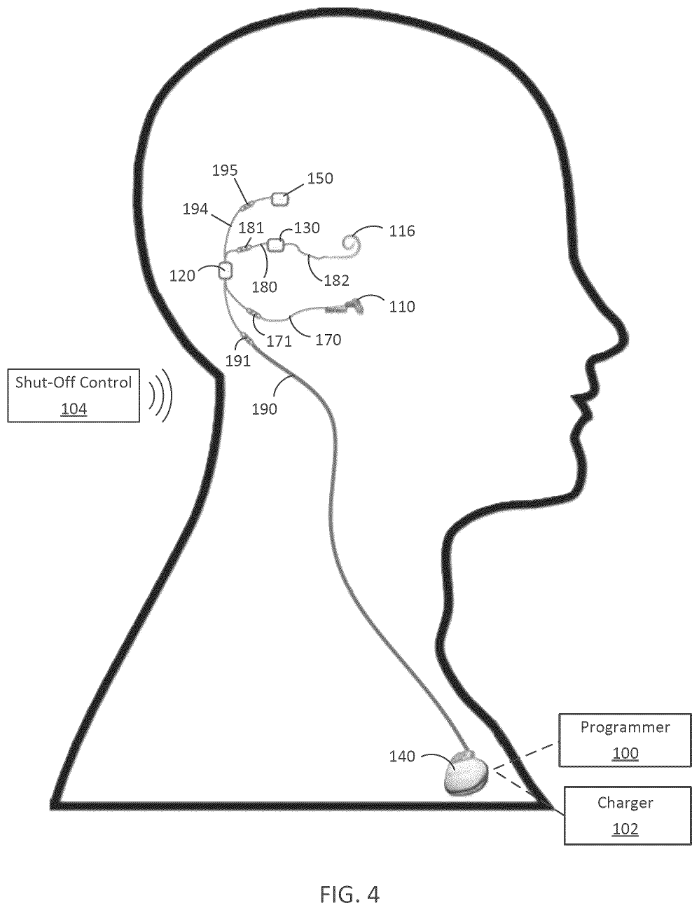

FIG. 4 is a schematic diagram illustrating an exemplary implantable system including an acoustic stimulator. The acoustic stimulator can be implanted proximate the patient's ossicular chain and can be in communication with a signal processor via lead 194 and detachable connector 195. The signal processor can behave as described elsewhere herein and can be configured to cause acoustic stimulation of the ossicular chain via the acoustic stimulator in in response to input signals from the middle ear sensor according to a transfer function of the signal processor.

The acoustic stimulator of FIG. 4 can be used similarly to the electrical stimulator as described elsewhere herein. For instance, an acoustic stimulator can be mechanically coupled to a patient's ossicular chain upon implanting the system and coupled to the signal processor via lead 194 and detachable connector 195. Similarly to systems described elsewhere herein with respect to the electrical stimulator, if the signal processor requires replacement or repair, the signal processor can be disconnected from the acoustic stimulator (via detachable connector 195) so that the signal processor can be removed without disturbing the acoustic stimulator.

In general, systems incorporating an acoustic stimulator such as shown in FIG. 4 can operate in the same way as systems described elsewhere herein employing an electrical stimulator and cochlear electrode only substituting electrical stimulation for acoustic stimulation.

Some systems can include a hybrid system comprising both an electrical stimulator and an acoustic stimulator in communication with the signal processor. In some such examples, the signal processor can be configured to stimulate electrically and/or acoustically according to the transfer function of the signal processor. In some examples, the type of stimulation used can depend on the input signal received by the signal processor. For instance, in an exemplary embodiment, the frequency content of the input signal to the signal processor can dictate the type of stimulation. In some cases, frequencies below a threshold frequency could be represented using one of electrical and acoustic stimulation while frequencies above the threshold frequency could be represented using the other of electrical and acoustic stimulation. Such a threshold frequency could be adjustable based on the hearing profile of the patient. Using a limited range of frequencies can reduce the number of frequency domains, and thus the number of contact electrodes, on the cochlear electrode. In other examples, rather than a single threshold frequency defining which frequencies are stimulated electrically and acoustically, various frequencies can be stimulated both electrically and acoustically. In some such examples, the relative amount of electrical and acoustic stimulation can be frequency-dependent. As described elsewhere herein, the signal processor transfer function can be updated to meet the needs of the patient, including the electrical and acoustic stimulation profiles.

Additionally or alternatively, while many examples show a middle ear sensor being in communication with an implanted signal processor, in various embodiments, one or more additional or alternative input sources can be included. For instance, in some embodiments, a microphone can be implanted under a user's skin and can be placed in communication with the signal processor (e.g., via a detachable connector such as 171). The signal processor can receive input signals from the implanted microphone and provide signals to the stimulator based on the received input signal and the signal processor transfer function. Additionally or alternatively, systems can include an inner ear sensor as an input source, wherein the inner ear sensor is configured to detect stimuli (e.g., pressure signals) from the wearer's inner ear (e.g., within the cochlear tissue).

With further reference to FIGS. 1 and 4, in some examples, a system can include a shut-off controller 104, which can be configured to wirelessly stop an electrical stimulator 130 from stimulating the patient's cochlear tissue and/or an acoustic stimulator 150 from stimulating the patient's ossicular chain. For example, if the system is malfunctioning or an uncomfortably loud input sound causes an undesirable level of stimulation, the user may use the shut-off controller 104 to cease stimulation from the stimulator 130. The shut-off controller 104 can be embodied in a variety of ways. For example, in some embodiments, the shut-off controller 104 can be integrated into other external components, such as the programmer 100. In some such examples, the programmer 100 includes a user interface by which a user can select an emergency shut-off feature to cease stimulation. Additionally or alternatively, the shut-off controller 104 can be embodied as a separate component. This can be useful in situations in which the patient may not have immediate access to the programmer 100. For example, the shut-off controller 104 can be implemented as a wearable component that the patient can wear at all or most times, such as a ring, bracelet, necklace, or the like.

The shut-off controller 104 can communicate with the system in order to stop stimulation in a variety of ways. In some examples, the shut-off controller 104 comprises a magnet that is detectable by a sensor (e.g., a Hall-Effect sensor) implanted in the patient, such as in the processor and/or the implantable battery and/or communication module 140. In some such embodiments, when the magnet is brought sufficiently close to the sensor, the system can stop stimulation of the cochlear tissue or ossicular chain.

After the shut-off controller 104 is used to disable stimulation, stimulation can be re-enabled in one or more of a variety of ways. For example, in some embodiments, stimulation is re-enabled after a predetermined amount of time after it had been disabled. In other examples, the shut-off controller 104 can be used to re-enable stimulation. In some such examples, the patient brings the shut-off controller 104 within a first distance of a sensor (e.g., a magnetic sensor) to disable stimulation, and then removes the shut-off controller 104. Subsequently, once the patient brings the shut-off controller 104 within a second distance of the sensor, stimulation can be re-enabled. In various embodiments, the first distance can be less than the second distance, equal to the second distance, or greater than the second distance. In still further embodiments, another device such as a separate turn-on controller (not shown) or the programmer 100 can be used to re-enable stimulation. Any combination of such re-enabling of stimulation can be used, such as alternatively using either the programmer 100 or the shut-off controller 104 to enable stimulation or combining a minimum “off” time before any other methods can be used to re-enable stimulation.

In some embodiments, rather than entirely disable stimulation, other actions can be taken, such as reducing the magnitude of stimulation. For example, in some embodiments, the shut-off sensor can be used to reduce the signal output by a predetermined amount (e.g., absolute amount, percentage, etc.). In other examples, the shut-off sensor can affect the transfer function of the signal processor to reduce the magnitude of stimulation in a customized way, such as according to frequency or other parameter of an input signal (e.g., from the middle ear sensor).

In some examples, implantable battery and/or communication module can be used to provide power and/or data (e.g., processing instructions) to other system components via lead 190. Different challenges exist for communicating electrical signals through a patient's body. For example, safety standards can limit the amount of current that can safely flow through a patient's body (particularly DC current). Additionally, the patient's body can act as an undesired signal path from component to component (e.g., via contact with the housing or “can” of each component).

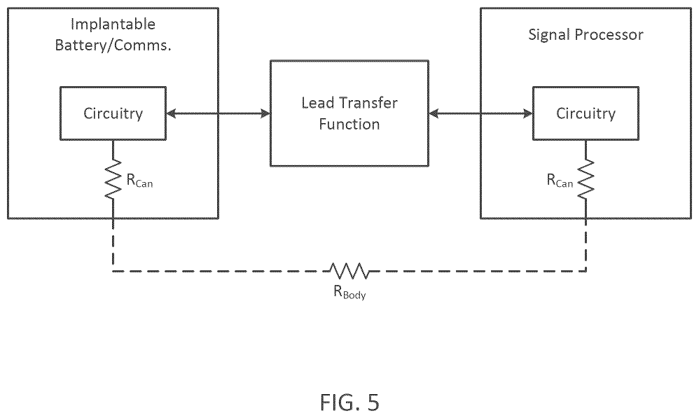

FIG. 5 is a high-level electrical schematic showing communication between the implantable battery and/or communication module and the signal processor. In the illustrated embodiment, the implantable battery and/or communication module includes circuitry in communication with circuitry in the signal processor. Communication between the circuitry in the implantable battery and/or communication module and the signal processor can be facilitated by a lead (190), represented by the lead transfer function. The lead transfer function can include, for example, parasitic resistances and capacitances between the leads connecting the implantable battery and/or communication module and the signal processor and the patient's body and/or between two or more conductors that make up the lead (e.g., 191). Signals communicated from the circuitry of the implantable battery and/or communication module to the circuitry in the signal processor can include electrical power provided to operate and/or stimulate system components (e.g., the middle ear sensor, signal processor, electrical and/or acoustic stimulator, and/or cochlear electrode) and/or data (e.g., processing data regarding the transfer function of the signal processor).

Various systems and methods can be employed provide communication between system components. Some examples of possible communication techniques are described in PCT patent application No. PCT/US20/19166, which is incorporated by reference. In some examples, data can be communicated to the implantable battery and/or communication module from an external component, such as a programmer as shown in FIG. 1. In an exemplary process, a programmer, such as a clinician's computer, can be used to communicate with a patient's fully implanted system via the implantable battery and/or communication module, which can communicate information to other system components, such as via lead 190.

During such processes, a clinician can communicate with the signal processor, and, in some cases, with other components via the signal processor. For example, the clinician can cause the signal processor to actuate an electrical and/or an acoustic stimulator in various ways, such as using various electrical stimulation parameters, combinations of active contact electrodes, various acoustic stimulation parameters, and various combinations thereof. Varying the stimulation parameters in real time can allow the clinician and patient to determine effectiveness of different stimulation techniques for the individual patient. Similarly, the clinician can communicate with the signal processor to update transfer function. For example, the clinician can repeatedly update the transfer function signal processor while testing the efficacy of each one on the individual patient. In some examples, combinations of stimulation parameters and signal processor transfer functions can be tested for customized system behavior for the individual patient.

In some embodiments, various internal properties of the system may be tested. For instance, various impedance values, such as a sensor impedance or a stimulator impedance can be tested such as described in U.S. Patent Publication No. 2015/0256945, entitled TRANSDUCER IMPEDANCE MEASUREMENT FOR HEARING AID, which is assigned to the assignee of the instant application, the relevant portions of which are incorporated by reference herein.

As described elsewhere herein, in various embodiments, the processor generally receives an input signal, processes the signal, and generates a stimulation signal, which can be applied via an integrated stimulator or a separate stimulator in communication with the processor (e.g., as shown in FIGS. 1 and 4). In some such embodiments, the input signal received via the signal processor is generated by an implantable sensor, such as a middle ear sensor.

However, such sensors often measure or otherwise receive some stimulus that is converted into an output that is read and processed by the signal processor. For example, some middle ear sensors may produce a different output signal for a given stimulus depending on a variety of factors, such as variability in a wearer's inner-ear anatomy and motion. Thus, the output of a sensor for a given input may be not predictable while designing a system, especially across a range of frequencies.

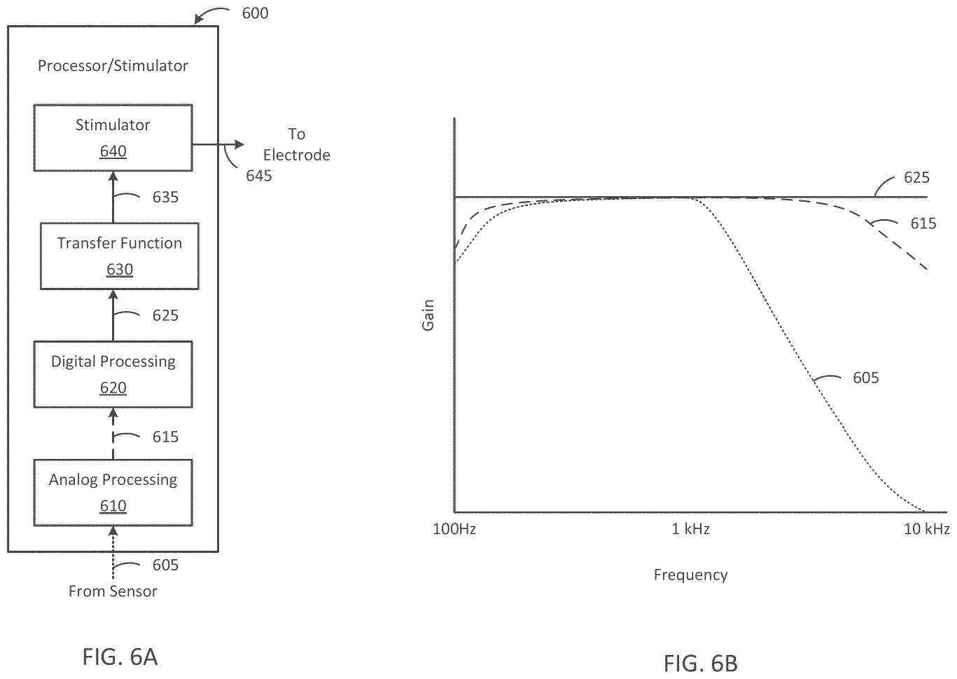

FIG. 6A is a schematic diagram showing an exemplary signal processing configuration for normalizing a stimulus signal and adapting to variability in a sensor frequency response. FIG. 6B shows an exemplary gain vs. frequency response curve for signals at various stages in the processing configuration. “Gain” associated with a particular frequency, as used with respect to FIG. 6B, refers to a relationship (e.g., a ratio) between the magnitude of an input stimulus received by the sensor and processor and the magnitude of the resulting signal at various stages of processing. In the illustrated example, the processor/stimulator 600 receives an input signal 605 from the sensor.

As shown in FIG. 6B, the gain is very uneven over the distribution of frequencies shown in the plot. For instance, according to the illustrated example, a stimulus signal received at the sensor at 1 kHz will result in a much larger magnitude in signal 605 compared to a stimulus signal of the same magnitude received at the sensor at 10 KHz. Such a discrepancy in frequency response can make signal processing difficult. Moreover, such frequency response in general may vary from person to person, or over the course of a wearer's lifetime due to physical movement of a sensor or anatomical changes.

The input signal 605 undergoes analog processing 610 to produce an analog processed signal 615. As shown in FIG. 6B, the analog processing step 610 improves the consistency of the gain across the range of frequencies, as the analog processed signal 615 provides a flatter frequency response curve than does the input signal 605. In some embodiments, the analog processing can include one or more filter and/or amplifiers generally configured to flatten out the frequency response curve as shown in FIG. 6B. In some examples, the analog processing components 610 within the processor/stimulator 600 can be substantially the same across various implantable systems in order to provide a first order correction of the frequency response. In other examples, an analog processing configuration 610 can be customized to the wearer, for example, based on known anatomical features, measurements, analysis, or the like.

The analog processed signal 615 undergoes a digital processing step 620 to produce a digitally processed signal 625. As shown in FIG. 6B, the digital processing step 620 further improves the consistency of the gain across the range of frequencies, as the digitally processed signal 625 provides a flatter frequency response curve than does the analog processed signal 615. In some embodiments, the digital processing 620 can be configured to substantially flatten the frequency response to correct remaining frequency response inconsistencies in the analog processed signal 615. For instance, in some embodiments, after digital processing 620, a stimulus signal of a given magnitude at a first frequency and a second frequency will result in a digitally processed signal 625 having the same magnitude at the first and the second frequencies. Thus, the digitally processed signal 625 corresponds to a normalized stimulus signal, reducing or eliminating the variability that comes with different wearer anatomies and wearer motion and/or changes over time. Having a normalized frequency response across large frequency ranges can simplify assessment of the efficacy of the implanted system, programming a signal processor transfer function, assessing system operation, and the like. In some examples, a flat frequency response can enable the system to present an electrical stimulus to the wearer at appropriate intensity levels, for example, with respect to received external acoustic stimuli, independent of the frequency content of the external acoustic stimuli.

In some embodiments, the digital processing 620 can be customized via a calibration process after the system has been implanted. In an exemplary calibration process, a clinician or other user may provide a series of stimulus signals, for instance, at a plurality of frequencies and having like amplitudes, to be “picked up” by the sensor, which generates an input signal 605 for each received signal. The clinician or other user may then sample the resulting analog processed signal 615 and/or an initial digitally processed signal 625 at the plurality of frequencies to determine the remaining non-uniformity in gain across the frequency sweep. The digital processing 620 can be either established or updated to compensate for non-uniformities in order to establish a substantially flat frequency response curve in the digitally processed signal 625. In some examples, a plurality of signals having different frequencies are provided in sequence and a magnitude response (e.g., gain) at each frequency is determined. After determining such a magnitude response, the digital processing stage 620 can be updated based on the response vs. frequency relationship in order to flatten the frequency response curve.

In an alternate process, a white noise signal can be provided to be “picked up” by the sensor. A transform (e.g., a Fast Fourier Transform, or FFT) of the signal can be performed in order to extract the frequency content of the signal. The extracted frequency content can used to determine a magnitude response at each frequency and the digital processing 620 can be updated to flatten the frequency response similar to described above.

In the illustrated example of FIG. 6A, the digitally processed signal 625 (e.g., having a uniform gain across a frequency range with respect to input signals received from the sensor) is processed according to the signal processor transfer function 630 to generate a stimulation signal 635. Stimulation signal 635 can be received by the stimulator 640, which can apply an electrical signal 645 to the electrode such as described elsewhere herein.

In some examples, the digital processing step 620 to provide a uniform frequency response can be incorporated into the transfer function 630 wherein the analog processed signal 615 is digitally processed to both flatten the frequency response and to generate a stimulation signal (e.g., 635) according to a programmed transfer function. Additionally or alternatively, as described elsewhere herein, in some examples, stimulator 640 can be located external to the processor rather than being combined as a single processor/stimulator component 600.

As described elsewhere herein, while many examples show a middle ear sensor being in communication with an implanted signal processor, in various embodiments, one or more additional or alternative input sources can be included. For instance, in some embodiments, a microphone can be implanted under a user's skin and can be placed in communication with the signal processor (e.g., via a detachable connector such as 171). The signal processor can receive input signals from the implanted microphone and provide signals to the stimulator based on the received input signal and the signal processor transfer function.

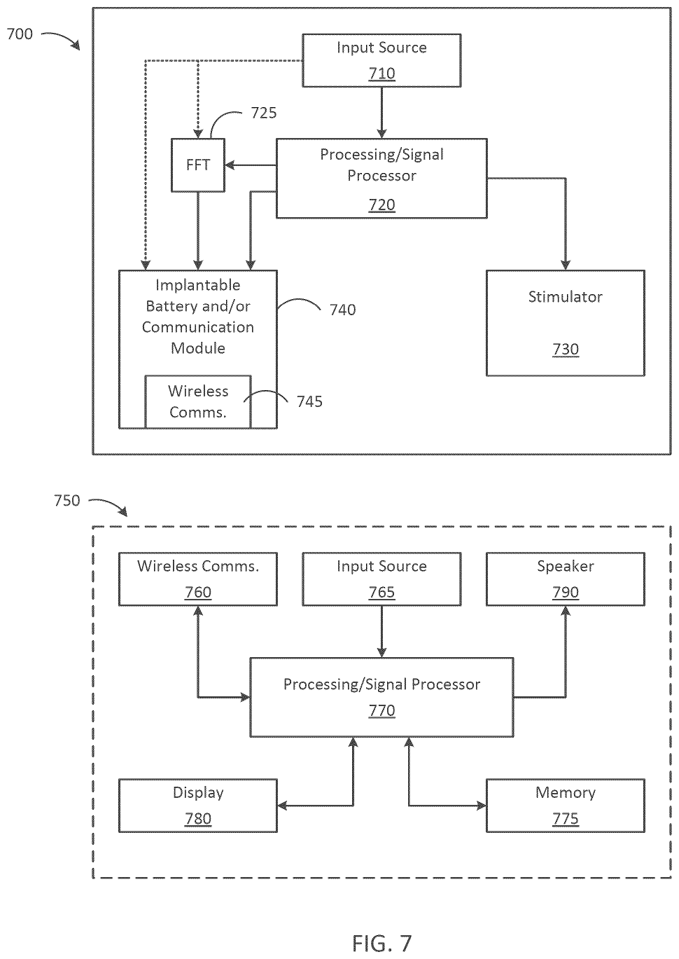

FIG. 7 shows a schematic diagram of an example cochlear implant and analysis system including an exemplary implantable cochlear implant system 700 and an external device 750. The implantable cochlear implant system (hereinafter “cochlear implant system”) 700 can be in communication with the external device 750 and can transmit signals to the external device 750. In an example operation, the external device 750 can receive and store signals from the cochlear implant system 700 while the cochlear implant system is operating. Further, the external device 750 can receive and store signals from an input source external to the cochlear implant system 700 while the cochlear implant system is operating. The external device 750 can optionally compare signals from the cochlear implant system with signals from the input source. In some examples, adjustments to the cochlear implant system 700 can be made based on the comparison of such signals.

The cochlear implant system 700 includes an input source 710, such as a microphone, middle ear sensor, inner ear sensor, or the like, in communication with signal processor 720. The input source 710 can be a sensor configured to receive an input representative of ambient sound and generate an input signal representative of the received input. For example, as described elsewhere herein, the input source 710 can be an inner ear sensor, middle ear sensor, microphone, or other sensor which can receive sound and generate a representative input signal. In operation, once generated, the input signal representative of the sound can be sent to the signal processor 720.

The signal processor 720 can be representative of any element or combination of elements which can receive an input signal from the input source 710 and generate an output signal based on the input signal. The generated stimulation signal can be based on the input signal but with additional processing. For instance, in some examples, the stimulation signal can be based on the input signal received by signal processor 720 and on a transfer function. In some examples, the signal processor comprises one or more additional processing components other than the signal processor which can perform further processing on the received input signals. In some examples, the processing performed by the signal processor 720 includes one or more intermediate steps used to generate the stimulation signal based on a signal received from input source 710. For instance, in some examples, one or more such intermediate steps include analog processing, digital processing, and/or generating a stimulation signal via a transfer function as described with respect to FIG. 6A and 6B.

In addition to being in communication with the input source 710, the signal processor 720 is in communication with a stimulator 730. As described elsewhere herein, the signal processor 720 can generate a stimulation signal which can be output to the stimulator 730 with the stimulation signal being used by the stimulator 730 to apply electrical signals to a patient's cochlear tissue via electrodes.

Further, signal processor 720 is in communication with an implantable battery and/or communication module 740. Power and/or data can be sent between the signal processor 720 and the implantable battery and/or communication module 740. In some examples, the implantable battery and/or communication module 740 sends power to the signal processor 720 and the signal processor 720 sends data to the implantable battery and/or communication module 740. In some examples, the implantable battery and/or communication module 740 sends both power and data to the signal processor 720.

In the example of FIG. 7, the signal processor 720 is additionally in communication with a transform stage 725. The transform stage 725 can be configured to transform a signal into a transformed signal. In some examples, the transform stage 725 is configured to perform a transform, such as a fast Fourier transform, to convert a signal into a frequency domain representation thereof. In FIG. 7, the transform stage 725 can receive signals (e.g., input signal) from the input source and/or the signal processor 720 and can output signals (e.g., transformed signal) to the implantable battery and/or communication module 740. While shown for illustrative purposes as being outside of the signal processor 720, in some embodiments, transformations performed by the transform stage 725 are performed by the same component(s) implementing functions of the signal processor 720.

While transform stage 725 is described above as applying a fast Fourier transform, in some examples, different transformations that result in an output signal comprising a frequency domain representation of the input signal can be used, such as a standard Fourier transform, a discrete Fourier transform, or other appropriate transform. In some embodiments, the transformation stage can be configured to transform a first signal into a frequency domain in squared units (e.g., V\hat{ }2 per frequency bin), and such squared units can be communicated to the external device 750 via the implantable battery and/or communication module 740.

In general, using transform stage 725 to transform the first signal into a frequency domain representation can enable the cochlear implant system to communicate data more efficiently. In some examples, transform stage 725 sample a signal for transformation (e.g., an input signal, an intermediate signal, etc.) for a predetermined period of time to generate an output signal. For instance, in some examples, the transform stage 725 can sample a first signal for an amount of time, for example, between 20 milliseconds and ten seconds, and perform a transformation (e.g., an FFT) on the sampled signal to generate the transformed signal representing the first signal. The transformed data can include data representing the entire span of time the signal was sampled, but can itself be a smaller data set when compared to transmitting the original signal for the entire duration of sampling. Thus, transforming the signal can enable the cochlear implant system to efficiently output a signal representative of the first signal to the external device 750. In some embodiments, transforming the signal can include averaging a transformed signal over several windows of time. For example, in some embodiments, transform stage 725 is configured to calculate a FFT of a desired signal over a predetermined time window. The transform stage 725 can calculate a plurality of FFTs of the signal over a corresponding plurality of time windows and average the FFTs in order to reduce the impact of random noise present in the signal.

Continuing with the example of FIG. 7, the cochlear implant system 700 can be configured to output one or more signals, such as an input signal from input source 710, a stimulation signal, and/or one or more intermediate signals, to an external device 750. In some examples, the input signal, the stimulation signal, and/or one or more intermediate signals are output by the cochlear implant system via the implantable battery and/or communication module 740. In such examples, the implantable battery and/or communication module 740 can receive an input signal, a stimulation signal, and/or one or more intermediate signals from one or more of the input source 710, the signal processor 720, and the transform stage 725. For instance, in some examples, the implantable battery and/or communication module 740 receives an input signal directly from the input source 710. Alternatively, in some examples, the implantable battery and/or communication module 740 can receive an input signal from the signal processor 720. In a further alternative, the implantable battery and/or communication module 740 can receive an input signal from the transform stage 725. In examples where the implantable battery and/or communication module 740 receives an input signal from the transform stage 725 or from the signal processor 720, the input signal can be a transformed and/or processed version of the input signal. In the example of FIG. 7, the implantable battery and/or communication module 740 receives a stimulation signal from the signal processor 720. However, as with the input signal, in some examples, the implantable battery and/or communication module 740 can receive a stimulation signal from the transform stage 725 which can be a transformed version of the stimulation signal.

In the example of FIG. 7, to output signals (e.g., an input signal and/or a stimulation signal) from the cochlear implant system 700 to the external device, the implantable battery and/or communication module 740 of the cochlear implant system 700 can include a wireless communication interface 745. The wireless communication interface 745 can communicate with the external device 750 which can include, in similarity with the cochlea implant system 700, a wireless communication interface 760. The wireless communication interface 745 of the cochlear implant system 700 can communicate with a wireless communication interface 760 of the external device 750 to exchange data such as input signals, stimulation signals, and/or other intermediate signals. While described as sending or outputting data to the external device 750, in some examples, the wireless communication interface 745 can receive signals from the external device 750 and can optionally send those signals to the signal processor 720. For example, some communication protocols may require sending data from the wireless communication module 760 in the external device 750 to the wireless communication module 760 in the implantable battery and/or wireless communication module. The wireless communication between the two wireless communication interfaces 745, 760 can comprise a variety of communication protocols, such as Wi-Fi, Bluetooth, NFC and/or other data transmission protocols.

While the example of FIG. 7 illustrates the wireless communication module 745 as part of the implantable battery and/or communication module 740, the wireless communication module 745 can be separate. For instance, in some examples, the wireless communication module can be in communication with the implantable battery and/or communication module but be located in a separate housing. Alternatively, in some examples, the signal processor 720 can include the wireless communication module and output signals to the external device 750 without going through the implantable battery and/or communication module.

In an example process of the cochlear implant system of FIG. 7, signal processor 720 can receive an input signal from input source 710 and generate a stimulation signal based at least in part on the input signal. The signal processor 720 can then output the stimulation signal to the stimulator for stimulation of a patient's cochlear tissue. The signal processor 720 can then output the stimulation signal and/or the input signal to the implantable battery and/or communication module 740 for outputting to the external device 750.

In another example process of the cochlear implant system of FIG. 7, the signal processor 720 still generates a stimulation signal based at least in part on an input signal received from the input source, but the input source 710 outputs the same input signal to the implantable battery and/or communication module 740 directly instead of through the signal processor 720. In such examples, the signal processor 720 can optionally output the stimulation signal to the implantable battery and/or communication module 740.

In some example operations, the wireless communication interface 745 of the cochlear implant system 700 can output an altered signal representative of the input signal and/or stimulation signal to the external device 750, such as a downsampled version or other variation of the input signal and/or stimulation signal to facilitate wireless communication.

Now specifically referencing the external device 750 of FIG. 7, the external device 750 includes a wireless communication interface 760, a second input source 765, a second signal processor 770, a memory 775, a display 780, and a speaker 790. While the illustrated embodiment includes the listed features, the external device 750 can be any device or series of devices that can receive signals from the cochlear implant system 700 and can store those signals in a memory. For example, the external device can be a computer, smartphone, tablet, or the like with a processor, a communication interface, and a memory. While a wireless communication interface is preferred, the external device can include a device that receives signals from the cochlear implant system through a wired communication interface.

As described elsewhere herein, the wireless communication interface 760 can communicate with the wireless communication interface 745 to send and/or receive signals. In similarity with the wireless communication interface 745 of the cochlear implant system 700, the wireless communication interface 760 of the external device 750 can be integrated in with the second signal processor 770 or alternatively, can be separate from and communicate with the second signal processor 770.

The second signal processor 770 of the external device 750 is in communication with the wireless communication interface 760 and can receive signals from the cochlear implant system 700 and/or send signals to the cochlear implant system 700 via the wireless communication interface 760. The second signal processor 770 can comprise one or more signal processors (e.g., digital signal processors), one or more microprocessors, microcontrollers, application specific integrated circuits (ASICs), or the like. In FIG. 7, the signal processor of the cochlear implant system can be considered a first signal processor 720 with the signal processor of the external device being considered the second signal processor 770. In addition to the wireless communication interface 760, the second signal processor 770 is in communication with a second input source 765, a memory 775, a display 780, and a speaker 790. In operation, the second signal processor 770 can receive signals from the wireless communication interface 760, the second input source 765, the memory 775, and the display 780 and can send signals to the wireless communication interface 760, the memory 775, the display 780, and the speaker 790. For example, the second signal processor 770 can receive a stimulation signal from the cochlear implant system 700 via the wireless communication interface 760 and can receive a second input signal from the second input source 765. The second signal processor 770 can then send the stimulation signal and the second input signal to the memory 775 to be stored.

The memory 775 of the external device 750 can receive and store signals sent from the second signal processor 770 such as a stimulation signal or first input signal from the cochlear implant system 700 or the second input signal from the external device 750. The memory 775 can comprise any type of computer memory including volatile memory, non-volatile memory, random access memory (RAM), read only memory (ROM), and the like. In some examples, the memory 775 comprises a non-transitory computer readable medium and can comprise instructions that can be executed by the second signal processor 770. In some examples, the memory is integrated with the second signal processor 770 while in some examples, the memory is separate from, but in communication with, the second signal processor 770.

Continuing with the example of FIG. 7, the external device 750 includes the second input source 765, which can comprise a microphone, middle ear sensor, inner ear sensor, or the like. The second input source 765 can be a sensor configured to receive an input representative of ambient sound and generate an input signal representative of the received input. For example, as described elsewhere herein, the second input source 765 can be an inner ear sensor, middle ear sensor, microphone, or other sensor which can receive sound and generate a representative input signal. In some examples, the second input source 765 of the external device can be considered a “second” input source with the input source 710 of the cochlear implant system 700 considered a “first” input source. In operation, once generated, the input signal representative of the sound can be sent to the second signal processor 770.

The external device 750 further includes a display 780 in communication with second signal processor 770. In some examples, the display 780 is separate from the external device 750 but is in communication with the external device 750 (e.g., via wireless communication interface 760). The display 780 can comprise any type of display (e.g., LCD, LED, OLED) and can include computer monitors, televisions, and handheld device displays.

The external device can also include a user interface to enable user input. For example, the display 780 of the external device can be a touch-sensitive display with a graphical user interface. A touch-sensitive display can allow a user to interact with the graphical user interface of the external device 750 and provide inputs to the external device 750. In some examples, the 3 external device can receive user inputs via a mouse, keyboard, and/or other human interface device. Inputs provided by a user to the external device 750 can control many different aspects of the external device including the wireless communication interface 760, the second input source 765, the second signal processor 770, the memory 775, the display 780, and the speaker 790.

The external device 750 of FIG. 7 also includes a speaker 790 in communication with second signal processor 770. In some examples, speaker 790 is separate from external device 750, but is in communication with the external device 750. Speaker 790 can be any type of speaker which produces audio output and can produce audio outputs representative of any signals which it receives. The speaker 790 can receive signals from the second signal processor 770 and generate an audio representation of the received signals. In some examples, the second signal processor 770 outputs a signal to the speaker to cause the speaker to output an audio representation of a given signal. For example, the speaker 790 can receive a signal from the second signal processor 770 and produce a pure tone, a phoneme, a word, a phrase, or other sound corresponding to the received signal.

While the wireless communication interface 760, the second input source 765, the memory 775, the display 780, and the speaker 790 are all illustrated as part of the external device 750, in some examples, one or more of the listed components are separate from the external device. In some such examples, the separate components are still in communication with the second signal processor 770 (e.g., via wired/wireless communication).

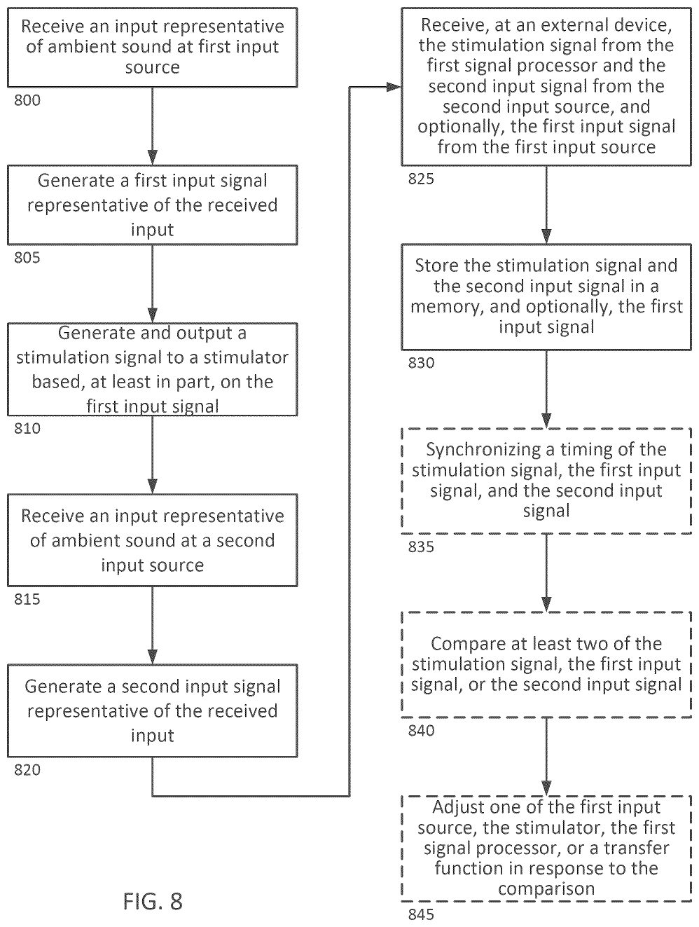

Now referencing both FIG. 7 and FIG. 8, FIG. 8 is a flow chart diagram of an example method of monitoring signals of an example cochlear implant and analysis system. The method of FIG. 8 is described with reference to the example cochlear implant and analysis system of FIG. 7, however a person having ordinary skill in the art will understand that the method of FIG. 8 is not limited to the illustrated structure of FIG. 7.

Starting with steps 800 through 810, which relate to the cochlear implant system 700 and its operation, the input source 710 (also “first input source”) receives a first input representative of ambient sound. The first input source 710 then generates a first input signal that is representative of the received first input. For example, the first input source 710 can be a middle ear sensor that receives an input in the form of vibrations of ear bones in response to ambient sound and generates a representative first input signal. In some examples, the ambient sound is generated by a speaker such as a speaker of an external device (e.g., speaker 790 of FIG. 7).The first signal processor 720 can then receive the first input signal from the first input source 710, generate a stimulation signal based at least in part on the first input signal, and output the stimulation signal a stimulator 730 to stimulate a patient's cochlear tissue. As described elsewhere herein, the first signal processor 720 can perform processing on the first input signal to generate the stimulation signal such that the stimulation signal can be based in part on a transfer function in addition to being based in part on the first input signal.

Continuing with steps 815 through 830, which relate to the external device 750 and its operation, the second input source 765 of the external device 750 receives ambient sound and generates a second input signal representative of the ambient sound. For example, the second input source 765 can be a microphone that receives ambient sound and generates the second input signal representative of the ambient sound. The ambient sound relating to the first input source 710 can be the same ambient sound received by the second input source 765. Continuing, the second signal processor 770 receives the stimulation signal that is generated by the first signal processor 720 such as described elsewhere herein. The second signal processor 770 further receives the second input signal from the second input source 765. In some examples, the second signal processor 770 can receive the first input signal generated by the first input source 710 in addition to or in lieu of the stimulation signal such as described herein. The second signal processor 770 then stores the stimulation signal, the second input signal, and optionally the first input signal in the memory 775.

Continuing with optional step 835, the second signal processor 770 can synchronize a timing of the stimulation signal, the second input signal, and optionally the first input signal. Synchronizing the timing of the signals can include adjusting a starting time of one or more of the stimulation signal, the second input signal, or the first input signal, for example, relative to an ambient sound. For instance, the first input signal is representative of an ambient sound, the stimulation signal is based at least in part on the first input signal which is representative of the ambient sound, and the second input signal is also representative of the ambient sound. The ambient sound can start at a start time and the second signal processor can adjust a timing of one or more of the first input signal, the stimulation signal, or the second input signal such that they align relative to each other with the start time of the ambient sound. Such synchronization can be used to compensate for delays in one signal relative to another due, for example, to data processing and/or communication steps.

Once the timing of the stimulation signal, the second input signal, and optionally the first input signal is synchronized, at least two of the stimulation signal, the first input signal, or the second input signal can be compared as in step 840. However, in some examples, the step of synchronizing a timing of the signals is not necessary to compare the signals. The comparison can include spectral analysis of the stimulation signal, the second input signal, and/or the first input signal. For instance, in some embodiments, the frequency content of each signal to be compared can be used in the comparison, such as the amount of each of a plurality of frequencies present in each signal. Frequency content can be extracted, for example, by performing a transform of the signal(s), such as an FFT. In various examples, comparing frequency content between two signals can include comparing the relative contributions of frequencies or ranges of frequencies to the compared signals. For example, frequency content of a signal can include a weighted intensity associated with each of a plurality of frequencies or range of frequencies. Additionally or alternatively, in some examples, an absolute magnitude of the frequency content of various frequencies or ranges of frequencies can be used for the comparison.

In some examples, the comparison is done manually such as by an audiologist. However, in some examples, the second signal processor can perform the comparison. In such examples, the second signal processor can use any type of comparison such as a frequency domain comparison.

In comparing the at least two of the stimulation signal, the second input signal, or the first input signal, the audiologist and/or the second signal processor can determine if the stimulation of a patient's cochlear tissue is representing the ambient sound as intended or desired. For example, the stimulation signal may differ significantly from the second input signal in a specific frequency range, despite both signals ultimately being based on the same ambient sound. Such a discrepancy can indicate that some portion of the cochlear implant system is not operating as desired.

After comparing the at least two of the stimulation signal, the second input signal, or the first input signal, adjustment to the cochlear implant system can be made in response to the comparison as in step 845. Adjustments to the cochlear implant system can include adjusting the first input source, the stimulator, the first signal processor, or a transfer function. For example, and audiologist can adjust one of the first input source, the stimulator, the first signal processor, or a transfer function (e.g., programmed into the first signal processor) to ensure a desired operation of the cochlear implant system. In some examples, a user can make adjustments to the cochlear implant system in response to the comparison. In some examples, the adjustment can be done automatically, such as by the first signal processor and/or the second signal processor.

For example, in some embodiments, if the frequency content of the first input signal is different from the frequency content of the second input signal, the first input source (e.g., 710) may be implanted or otherwise arranged in such a way that its frequency response is unexpected or non-uniform. In response, the first input source could be adjusted or replaced (e.g., surgically). In other examples, such non-uniformity and/or unexpected operation can be corrected by analog and/or digital processing of the first input signal, such as described in U.S. Patent Publication No. 2020/0269035 A1, entitled IMPLANTABLE COCHLEAR SYSTEM WITH INTEGRATED COMPONENTS AND LEAD CHARACTERIZATION, which is hereby incorporated by reference. In some examples, a signal processor 720 transfer function can be updated to compensate for detected discrepancies, such as by increasing or decreasing gain at one or more frequencies based on the comparison.

For instance, in an example embodiment, if frequency content of the first input signal in a first range of frequencies is comparatively low relative to the frequency content of the second input signal in the first frequency range, the signal processor transfer function can be adjusted to increase a gain in the first frequency range to compensate for comparatively lower amount of such frequencies in the first input signal. Similarly, in another example, the signal processor transfer function can be updated to increase a gain in a first range of frequencies if the frequency content of the stimulation signal in the first range of frequencies is comparatively low relative to the frequency content of the second input signal in the first range of frequencies (e.g., due to reduced frequency content in the first input signal used to generate the stimulation signal and/or the processing techniques used to generate the first input signal).

In some embodiments, adjusting the transfer function can be performed by an audiologist analyzing the signals (e.g., the second input signal and the stimulation signal). In other examples, adjustments can be performed using, for example, artificial intelligence or a neural network trained to analyze differences in signals (e.g., the second input signal and the stimulation signal) and update a signal transfer function to better match the signals (e.g., to minimize differences between a first and second input signal and/or to make a stimulation signal match a desired stimulation signal in response to a received acoustic stimulus).

In some embodiments, processes similar to the process of FIG. 8 can be performed in response to initiation by a wearer of the cochlear implant system. In some embodiments, a wearer can, via an external device in wireless communication with an implanted cochlear implant system, initiate such a process, for example, via a smartphone app. For example, a user may have difficulty hearing or perceiving certain sounds and/or sounds in certain environments, and may initiate such a process to help improve the functionality of the cochlear implant system.

In some such examples, upon initiating such a process, the external device (e.g., a smartphone) can record environmental acoustic information as well as stimulation data and/or sensor input data such as described elsewhere herein. In various embodiments, the external device is configured to save the data in a memory. In some examples, the external device can save such data in a local memory. Additionally or alternatively, in some embodiments, the external device can communicate the data to another location, such as saving the data to a cloud-based storage location. In some examples, the external device can save and/or send the data for viewing by an audiologist, such as by syncing, uploading, or emailing the information, for example. Additionally or alternatively, in some examples, a user can bring the external device to the audiologist for data transfer and/or analysis.

As described herein, in some embodiments, the external device is configured to synchronize the data streams (e.g., an input signal representative of an acoustic environment and a stimulation signal generated based on the input signal). In other examples, the data can be synchronized at another time and/or location, such as after it has been communicated to an audiologist.

Additionally or alternatively, in some examples, additional information representing an environment can be observed, such as an external environment of a wearer and/or an environment of an implanted cochlear implant system. For instance, in some examples, a device (e.g., an external device) comprises or is otherwise in communication with a pressure sensor configured to detect an ambient pressure of the wearer's environment and/or an altimeter configured to detect an altitude of the wearer from which other environmental factors can be determined. Additionally or alternatively, an implanted pressure sensor can detect changes within a wearer's body.

Changes in such environmental (internal and/or external) conditions can affect the operation of a cochlear implant system. For instance, changes in external pressure (e.g., due to altitude changes, traveling by airplane, etc.) or internal pressure (e.g., due to illness and/or Estuation tube disfunction) can affect operation of a middle ear sensor configured to detect motion of one or more middle ear structures in response to acoustic stimulus.

In some embodiments, a pressure change can be detected, for example, by the signal processor of the cochlear implant system and/or by an external device. In some examples, detecting a pressure change comprises detecting a change in pressure by at least a predetermined amount (e.g., an absolute amount or a percentage). Additionally or alternatively, in some examples, detecting a pressure change comprises detecting a pressure value crossing above or below a predetermined threshold.

In some examples, if a pressure change is detected, the signal processor and/or external device can adjust a transfer function based on the pressure change, for example, by implementing a transfer function associated with an absolute pressure measurement or associated with a detected change in pressure. Additionally or alternatively, in some examples, the external device can alert the wearer of a detected pressure change and inform that wearer that performance of the cochlear implant system may temporarily change due to the pressure change. This allows the wearer, if experiencing a change in system operation (e.g., changed sound perception) due to such pressure, to understand the reason. The external device can also suggest that the user contact a provider (e.g., an audiologist) if the change does not resolve, for example, within a predetermined period of time (e.g., within one week).

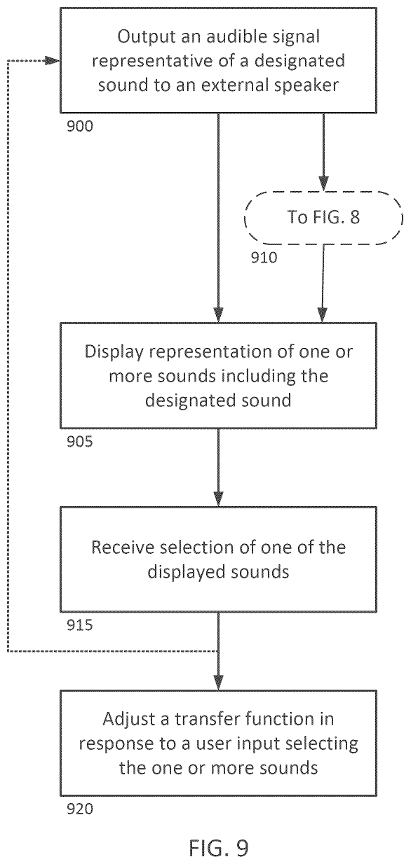

FIG. 9 is a flow chart diagram of another example method of monitoring signals of an example cochlear implant and analysis system. The method starts in step 900 by outputting an audible signal representative of a designated sound to an external speaker (e.g., 790). In various examples, the designated sound can be selected automatically by the system, such as a pre-set sound for use in a process such as described herein. In some examples, the sound can be selected manually, such as by an audiologist conducting or overseeing a process such as those described herein. In some examples, the audible signal is representative of a selected phoneme, pure tone, word, or phrase. In some examples, the audible signal is representative of a combination of a selected phoneme, pure tone, word, or phrase. In some embodiments, a designated sound can include a consonant-nucleus-consonant word (“CNC word”).

In step 905, a graphical representation of one or more sounds, including the designated sound of step 900, is displayed. In some examples, the one or more sounds are displayed on a display connected to and/or part of an external device (e.g., 780). Similar to those discussed above with respect to step 900, in some examples, the display can present graphical representations of one or more phonemes, pure tones, words, or phrases, or, for example, words or phrases incorporating or describing the sounds, such as a plurality of words incorporating different phonemes, one of which corresponds to the designated sound.

The method then continues with step 915, which includes receiving a selection of one of the displayed sounds. In some examples, a user can select one of the displayed sounds via an interface, such as a touchscreen or other interface capable of receiving a user input. In some examples, after the selection is received, the process can repeat with one or more additional designated sounds.

The method continues with step 920 and includes adjusting a transfer function in response to a user input selecting the one or more sounds displayed in step 905. For example, a user can select one phoneme from a list of one or more phonemes displayed in step 905 and, based on the selection, the transfer function can be adjusted accordingly. As discussed elsewhere herein, the transfer function can be used with, or as part of, a signal processor (e.g., 720). In some examples, the transfer function is adjusted automatically via one or more signal processors (e.g., 720). Additionally or alternatively, in some examples, the transfer function is adjusted manually, such as by an audiologist.

In some cases, adjusting the transfer function improves the user's perception via the cochlear implant. For instance, in an illustrative example embodiment of the process of FIG. 9, a phoneme (e.g., “va”) can initially be selected. Starting with step 900, an audible signal representative of the selected phoneme of “va” can be output by an external speaker (e.g., a phone speaker). Next, in step 905, a graphical representation of the selected phoneme of “va” can be displayed alongside representations of other phonemes including, for example, “sa” and “cha” on a display (e.g., a phone display). Such additional phonemes can be ones similar to, but different than the selected phoneme. Next, in step 915, a user can choose one of the phonemes of “va”, “sa”, and “cha” that are displayed. If the user chooses either of the phonemes “sa” or “cha”, which were not the selected phoneme of “va” originally output to the external speaker, a transfer function of the cochlear implant system (e.g., of the first signal processor) can be adjusted to assist a wearer in better identifying the phoneme.

Adjusting a transfer function can enable a user to ensure that they are hearing the correct phoneme output by an external speaker. The process can be repeated for many different phonemes to increase a user's speech recognition. Additionally, while phonemes are described in this illustrative embodiment, as described elsewhere herein, other sounds, such as pure tones, words (e.g., CNC words), or phrases can be used.

In some embodiments, adjusting a transfer function comprises analyzing a frequency content of the designated sound and the representation chosen by the user. For example, in some embodiments, confusion between the designated sound output acoustically and the particular presented sound chosen by the user suggests one or more specific transfer function updates that can be performed to reduce the likelihood of confusion, such as by increasing or decreasing the gain associated with one or more frequencies. For instance, in an example embodiment, mistaking one particular phoneme (e.g. “va”) for another (e.g., “sa”) can be correlated to a particular frequency or set of frequencies, such as one or more harmonics of a fundamental frequency. In some embodiments, frequency content of phonemes used to assess confusion can be stored in memory, for example, in order to identify, when certain phonemes are confused for one another, which frequency content may be contributing to the confusion (e.g., frequencies that, if perceived, would improve the ability to distinguish between the phonemes).

For example, an “s” sound typically has higher frequency content than a “v” sound even if fundamental frequency content of the two sounds are similar. Accordingly, an ability or inability to distinguish between the two phonemes can be correlated with perceiving harmonics of the fundamental frequency. If a wearer has difficulty differentiating between “va” and “sa,” for example, adjusting a transfer function to boost gain in the harmonic frequencies can help a wearer perceive such harmonics and distinguish between the phonemes. In some examples, after determining phonemes between which a wearer has difficulty distinguishing, differences in frequency content of those phonemes (e.g., differences in harmonics) can be used to guide a change in a transfer function to assist the wearer in better distinguishing between such phonemes.

Additionally or alternatively, in some embodiments, adjusting a transfer function comprises adjusting stimulation on a per-electrode basis. For instance, as described elsewhere herein, in some embodiments, electrical stimulation can be provided via a cochlear electrode having a plurality of contact electrodes, each in contact with a different portion of a wearer's anatomy. In some embodiments, updating a transfer function comprises adjusting a magnitude and/or location (e.g., contact electrode or set of contact electrodes) of electrical stimulation provided for a given input signal.

In some embodiments, adjusting the transfer function comprises consulting a confusion matrix that can include, for example, transfer function adjustments associated for each pair of the sound output from a speaker and the sound chosen on the display by the user. Such a confusion matrix can be populated by using past fitting data, for example, shown to improve distinction between the two sounds (e.g., phonemes). In some examples, such a confusion matrix can be stored in a database (e.g., an internet-accessible database) and generated from data gathered from a plurality of wearers. In other examples, a confusion matrix can be generated for an individual wearer, such as by an audiologist adjusting a transfer function (e.g., a gain associated with one or more frequencies and/or a stimulation location associated with an input signal) and repeating a played sound to see if the user's perception of the particular sound has improved (e.g., evidenced by the user selecting a correct representation of the played sound on a display).

As noted above, in some examples, steps 900, 905, and 915 are performed for a plurality of sounds, such as a plurality of phonemes or a plurality of CNC words. In some examples, the plurality of sounds for which such steps are performed need not be the same type of sound. For example, in some embodiments, a first phoneme can be used as a first designated sound for which steps 900, 905, 915 are performed and a first CNC word can be used as a second designated sound for which such steps are also performed.

As shown in the example, of FIG. 9, in some embodiments, a plurality of designated sounds can be output to a speaker (900) and a corresponding plurality of received selection of displayed sounds can be received (915) prior to adjusting a transfer function (920). However, in some embodiments, step 920 can be performed after each time a sound is output to an external speaker (900) and selection is received (915) such that the transfer function is updated a plurality of times if a plurality of designated sounds are used. In various examples, the transfer function is updated based only on the received selection and the output designated sound immediately preceding the updating. In other examples, updating the transfer function accounts for multiple previous pairs of sounds output in step 900 and selections received in step 915.

It will be appreciated that, in some examples, the process of FIG. 8 can be performed in conjunction with one or more steps in the process of FIG. 9. For example, in some cases, after step 900 in FIG. 9, the process of FIG. 8 (represented at step 910 in FIG. 9) can be performed using the audible signal output to the external speaker. In various examples, if step 910 (the process of FIG. 8) is performed, it can be performed before proceeding to step 905, after proceeding to step 905, or in parallel with proceeding to step 905. If the method of FIG. 8 is performed at step 910, optional steps of 840, 845, and 850 are still optionally performed such as described above with respect to FIG. 8.

In some examples in which the processes of FIGS. 8 and 9 are both performed, adjusting a transfer function at step 915 and at step 845 can be performed as a single adjustment step. For example, a transfer function can be updated to compensate for detected inconsistencies between a first and second input signal by adjusting a gain associated with one or more frequencies such as described with respect to FIG. 8, and additionally can be updated in such a way so as to improve the recognition of one or more sounds, such as one or more phonemes or CNC sounds such as described with respect to FIG. 9.

In some examples, an external device is configured to instruct a user to perform a process similar to that shown in FIG. 9 and/or to guide a user through performing such a process, such as if a user manually initiates the process. In some examples, a cochlear implant system includes an external speaker calibrated for outputting desired sounds, such as particular tones, words, phonemes, etc. In some such examples, an external device can instruct the user to position the external speaker in an appropriate position relative to the user, such as instructing the user to place the speaker in front of them and continue with the procedure. The calibrated speaker can be used to output the audible signal of step 900 of the process in FIG. 9.

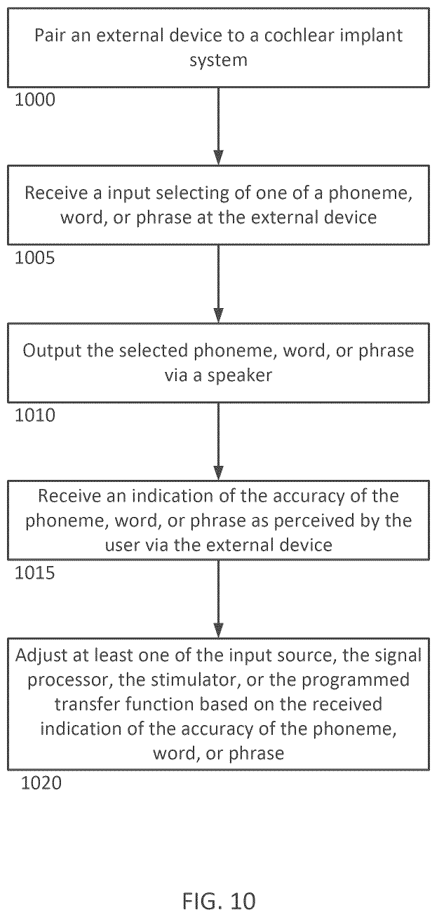

In some examples, a user can interface with the system to manually test the user's perception of one or more sounds. FIG. 10 is a flow chart diagram of an example method of analyzing signals of an example cochlear implant and analysis system. Starting with step 1000, an external device (e.g., 750) is paired to a cochlear implant system (e.g., 700). As described elsewhere herein, the external device can be paired to the cochlear implant system via Bluetooth or other wireless communication protocols using wireless communication interfaces of the external device and the cochlear implant system. Once paired, a selection of a phoneme, word, or phrase can be received at the external device as in step 1005. For example, a user can provide an input to an external device via a user interface in communication with the external device designating a sound. The input can include selecting from a predefined list of sounds, such as words, phonemes, or tones.

The selected phoneme, word, or phrase is then output from the external device via a speaker as in step 1010, where it can be received by the user (e.g., picked up by an input source of the user's cochlear implant system). For example, the speaker of the external device can audibly output a word “bat” in response to the user selecting such a word via a user interface.

Next, in step 1015, the external device can receive an indication of the accuracy of the phoneme, word, or phrase as perceived by the user. For example, the user can hear the phoneme, word, or phrase output via the speaker and subsequently provide an input to the external device that indicates the accuracy of the phoneme, word, or phrase the user perceived. In one such example, the user can input to the external device whether they clearly perceived the selected sound and/or an input indicative of the sound they did perceive. For example, if the external device causes the speaker to say the word “bat,” the user may provide an input to the external device that they perceived the word “pat,” indicating inaccuracy. In other examples, the user may rate the accuracy of the perceived sound, such as on a scale of 1-10.

Further, in step 1020, the method includes adjusting at least one of the input source, the signal processor, the stimulator, or a programmed transfer function of the signal processor, based on the received indication of the accuracy of the selected phoneme, word, or phrase. For example, the programmed transfer function can be adjusted to emphasize certain frequencies over others to ensure the user perceives the word “bat” rather than the word “pat” when “bat” is output by the speaker. In some examples, such adjustments can be implemented automatically via the external device. For instance, in some embodiments, the external device can analyze the indication of the accuracy of the sound as perceived by the user and update the transfer function of a signal processor of the user's cochlear implant system accordingly, such as to adjust a gain associated with one or more frequencies.

In some embodiments, the external device logs one or more indications of the accuracy of the sound as perceived by the user. In some such examples, the external device can update operation of the cochlear implant system based on one or more saved indications of accuracy. Additionally or alternatively, the user may initiate and perform the process of FIG. 10 with one or more sounds before providing the accuracy log to an audiologist for analysis and/or updating the operation of the cochlear implant system such as by updating a transfer function.

As described, in some examples, known confusion between sounds (e.g., phonemes, words, etc.) can be used to update a transfer function to assist a wearer in differentiating between such sounds. Various processes described herein can be used to establish areas of confusion, for example, creating a confusion matrix. In some cases, confusion can be analyzed by examining synchronized data. For example, as described elsewhere herein, in some embodiments, various data can be logged and synchronized for comparison.

In some embodiments, an external device (e.g., a smartphone, laptop, etc.) can be configured to output one or more sounds that are perceived at an input source of a cochlear implant system. A signal processor of the cochlear implant system can generate a stimulation signal based on an input signal generated by the input source in response to the perceived sound. An external microphone can also pick up the sound(s) output from a speaker to produce a second input signal based on the sound picked up at the microphone.

As described elsewhere herein, in some cases, a stimulation signal generated in the cochlear implant and the second input signal generated by the external microphone can be synchronized and compared. In some examples, such a comparison can be performed in response to one or more identified words or phonemes that are difficult for a wearer to differentiate. In some examples, frequency content of such signals are compared for a like time period, such as during a time period when a predetermined sound (e.g., predetermined phoneme) is output from a speaker.

As described elsewhere herein, in some cases, different phonemes may have similar fundamental frequency content, but differ in one or more harmonics. Frequency analysis of, for example, the second input signal (from the external microphone) and the stimulation signal may reveal certain frequencies (e.g., one or more harmonics) that are present in the second input signal but lacking or suppressed in the stimulation signal. For instance, mechanical coupling of a middle ear sensor to a middle ear structure and/or middle ear anatomy itself may vary from person to person in a way where some frequencies are not output by the middle ear sensor as strongly as other frequencies.

Discrepancies in frequency content of the second input signal and the stimulation signal can contribute to a wearer's difficulty differentiating between certain phonemes where the differences between such phonemes present within those frequencies. In some cases, a frequency analysis is performed based on and/or in response to the results of one or more tests to determine whether or which phonemes present confusion to a wearer. Such a frequency analysis can confirm a source of likely confusion, for example, based on discrepancies in certain frequencies (e.g., one or more harmonics) between a second input signal (from an external microphone) and a stimulation signal.

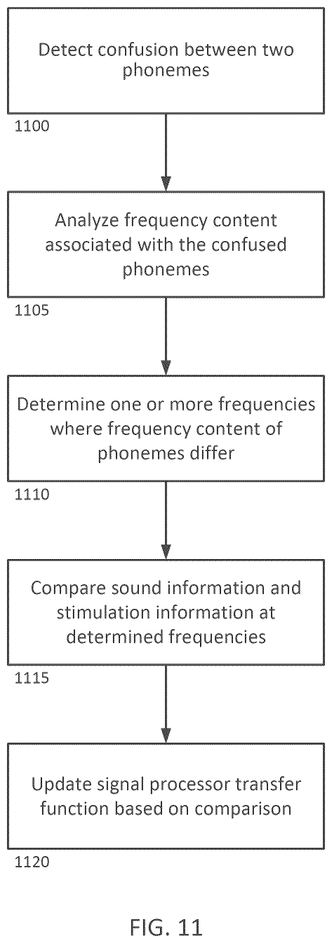

FIG. 11 is a flow chart diagram of an example method of updating a transfer function based on detected confusion between phonemes. In the illustrated example, the process starts by detecting confusion between two phonemes at step 1100. This can be performed, for example, via a confusion matrix, by directly testing an ability to distinguish between such two phonemes, or other processes. The process further includes analyzing frequency content associated with the confused phonemes at step 1105 and determining one or more frequencies where frequency content of the phonemes differ at step 1110. In some cases, frequency content associated with the phonemes are stored in memory for analysis.

The process includes comparing sound information (e.g., as provided by an external microphone) and stimulation information (e.g., as generated by a cochlear implant signal processor) at the determined frequencies where the frequency content of the phonemes differ at step 1115. Differences in the sound information and stimulation information at such frequencies can demonstrate that the stimulation signal within the cochlear implant system is not large enough in those frequencies that distinguish the phonemes.

The process further includes updating a signal processor transfer function based on the comparison of the sound information and stimulation information at step 1120. For example, if the stimulation information has, compared to the sound information, lower frequency content in one or more frequencies where the frequency content phonemes differ, a wearer may have difficulty distinguishing between such phonemes, since the stimulation signal in the distinguishing frequencies is low. Updating the signal processor transfer function can include increasing a gain at such frequencies so that the frequencies are perceived more strongly by the wearer. Since such frequencies are different between the confused phonemes, stronger stimulation at such frequencies can help the wearer differentiate between the phonemes.

Various processes described herein can be embodied in a non-transitory computer readable medium, for example, that can be implemented via a smartphone app, PC software or other suitable devices. In an example implementation, a non-transitory computer readable medium is configured to cause one or more programmable processors to perform one or more methods for identifying and/or addressing confusion. Methods can include presenting one or more sounds to a user via a speaker and assess the perception of the sounds, for example, by playing a word or phoneme via a speaker and providing a user with a multiple choice selection of words or phonemes including one correct answer, corresponding to the word or phoneme output via the speaker, and one or more incorrect answers. Other processes of determining confusion are possible. Methods can further include collecting and storing confusion information regarding whether one or more sounds caused confusion by a wearer, for example, by storing information in a confusion matrix.

Methods can include storing sound information received from an external microphone (e.g., as part of a smartphone, PC, or the like) and stimulation information received from a cochlear implant system (e.g., a representation of a stimulation signal) and synchronizing the sound information and stimulation information. Methods can further include analyzing the synchronized sound and stimulation information in view of the stored confusion information. For example, methods can include identifying an area of confusion (e.g., one phoneme confused for another), identifying one or more frequencies or frequency ranges associated with the confusion (e.g., frequency content differences between confused phonemes, such as one or more harmonic frequencies), and analyzing the difference in frequency content between the stimulation information and the sound information in the identified frequencies or frequency ranges to confirm the source of confusion. Analyzing the difference in frequency content can include, for example, comparing the frequency content at one or more frequencies or frequency ranges. Additionally or alternatively, in some examples, methods include presenting information regarding one or more identified frequencies or frequency ranges associated with the confusion via an interface (e.g., a display) for analysis by a human.

The frequency information analysis, such as comparing frequency information at one or more frequencies or frequency ranges, can be used to confirm a source of confusion between sounds. For example, a noted difference in certain frequencies between sound and stimulation information can confirm a source of confusion between phonemes having frequency content that differ in such frequencies.

In some examples, such information can be used to adjust the transfer function of the cochlear implant system signal processor to reduce or eliminate the difference in frequency content between the sound and stimulation information. For instance, if the frequency content of the stimulation signal in one or more frequencies associated with identified sound confusion is suppressed compared to the corresponding frequency content of the sound used to generate the stimulation signal (e.g., the sound picked up by a cochlear implant input source), the transfer function can be updated to increase gain associated with such frequencies such that the frequency content of the stimulation signal is increased.

In some examples, an external device such as a smartphone or PC can be configured to interface with the cochlear implant (e.g., via a wireless communication interface such as Bluetooth) to update the transfer function of the signal processor based on the frequency analysis. In some cases, the external device is configured to recognize relevant frequencies based on confusion information, for example, determining a wearer has difficulty distinguishing between “va” and “sa” phonemes and determining frequencies related to such phonemes (e.g., harmonics of a fundamental frequency). The external device can analyze frequency content in sound information and stimulation information in those determined frequencies (e.g., over a time period during which a test phoneme was presented to the wearer via a speaker). If the frequency content is different in one or more relevant frequencies, the external device can act to update a transfer function of the cochlear implant system signal processor based on the difference. For instance, if a harmonic of a fundamental frequency is suppressed in the stimulation information frequency content compared to in the sound information, the external device can update the transfer function to increase a gain associated with such frequency.

While in some examples, all such steps can be performed automatically via the external device (e.g., a smartphone or PC comprising a non-transitory computer readable medium including instructions for causing a programmable processor carry out such steps), in some cases, a user interfacing with the external device can perform one or more steps.

Various non-limiting embodiments have been described. These and others are within the scope of the following claims.

1. A cochlear implant and analysis system comprising:
an implantable cochlear implant system comprising:
a stimulator configured to provide electrical stimulation;
a first input source configured to receive a first input representative of an ambient sound and generate a first input signal representative of the received first input; and
a first signal processor in communication with the stimulator and the first input source, the first signal processor being programmed such that the first signal processor generates and outputs a stimulation signal to the stimulator based, at least in part, on the first input signal received from the first input source;
a second input source configured to receive the ambient sound and generate a second input signal representative of the ambient sound;
an external device in wireless communication with the implantable cochlear implant system and in communication with the second input source, the external device comprising a second signal processor, the second signal processor being programmed to:
receive the stimulation signal generated by the first signal processor;
receive the second input signal from the second input source; and
store the stimulation signal and the second input signal in a memory.

2. The cochlear implant and analysis system of claim 1, further comprising an implantable battery and/or communication module in communication with the first signal processor, the second signal processor being programmed to receive the stimulation signal generated by the first signal processor via the implantable battery and/or communication module.

3. The cochlear implant and analysis system of claim 1, wherein the second signal processor is further configured to receive and store the first input signal from the first input source.

4. The cochlear implant and analysis system of claim 3, wherein the second signal processor is configured to synchronize and store the stimulation signal, the first input signal, and the second input signal together in the memory such that they are synchronized in time.

5. The cochlear implant and analysis system of claim 4, wherein:
the first signal processor is programmed with a transfer function such that the stimulation signal is further based on the transfer function; and
the second signal processor is programmed to:
compare at least two of the first input signal, the second input signal, or the stimulation signal; and
update the transfer function in response to comparing the at least two of the first input signal, the second input signal, or the stimulation signal.

6. The cochlear implant and analysis system of claim 1, wherein the second signal processor is configured to synchronize and store the stimulation signal and the second input signal together in the memory such that they are synchronized in time.

7. The cochlear implant and analysis system of claim 6, wherein:
the first signal processor is programmed with a transfer function such that the stimulation signal is further based on the transfer function; and
the second signal processor is programmed to:
compare the stimulation signal and the second input signal; and
update the transfer function in response to comparing the stimulation signal and the second input signal.

8. The cochlear implant system of claim 7, wherein the second signal processor is configured to:
receive information indicative of confusion between at least two phonemes; and
determine one or more frequencies associated with the at least two phonemes; and wherein
comparing the stimulation signal and the second input signal comprises comparing the frequency content of the stimulation signal and the second input signal in the one or more frequencies.

9. The cochlear implant system of claim 8, wherein receiving information indicative of confusion between at least two phonemes comprises receiving a response via a user interface indicative of a phoneme perceived by a user in response to being prompted with one or more phonemes.

10. The cochlear implant and analysis system of claim 1, wherein the external device is configured to cause an external speaker to output a sound corresponding to a selected phoneme such that the ambient sound comprises the selected phoneme output from the external speaker.

11. The cochlear implant and analysis system of claim 1, wherein the second input source is part of the external device.

12. The cochlear implant and analysis system of claim 1, wherein the second signal processor is configured to receive the stimulation signal from the first signal processor and the second input signal from the second input source simultaneously.

13. The cochlear implant and analysis system of claim 1, wherein:
the first input source comprises a first microphone, the first input comprising the ambient sound;
the second input source comprises a second microphone; and
the external device comprises a personal computing device.

14. The cochlear implant and analysis system of claim 1, wherein:
the first input source comprises a middle ear sensor; and
the first input comprises vibrations from at least one of a patient's tympanic membrane, malleus, incus, or stapes.

15. The cochlear implant and analysis system of claim 1, wherein the stimulation signal received from the first signal processor comprises a plurality of stimulation channels, the plurality of stimulation channels corresponding to a plurality of contact electrodes electrically connected to the stimulator.

16. A method of monitoring signals for a cochlear implant system comprising:
receiving a first input representative of an ambient sound at a first input source;
generating a first input signal representative of the received first input;
generating and outputting, via a first signal processor, a stimulation signal to a stimulator based at least in part on the first input signal received from the first input source;
receiving the ambient sound at a second input source;
generating, via the second input source, a second input signal representative of the ambient sound;
receiving, at an external device, the stimulation signal generated by the first signal processor and the second input signal generated by the second input source; and
storing the stimulation signal and the second input signal in a memory.

17. The method of claim 16, further comprising:
receiving, at the external device, the first input signal from the first input source; and
storing the first input signal in the memory.

18. The method of claim 17, wherein storing the stimulation signal, the first input signal, and the second input signal in the memory further comprises synchronizing a timing of the stimulation signal, the first input signal, and the second input signal.

19. The method of claim 17, further comprising:
comparing at least two of the stimulation signal, the first input signal, or the second input signal; and
adjusting one of the first input source, the stimulator, the first signal processor, or a transfer function in response to the comparison of the at least two of the stimulation signal, the first input signal, or the second input signal.

20. The method of claim 19, wherein the comparing comprises comparing spectral components of the at least two of the stimulation signal, the first input signal, or the second input signal, over time.

21. The method of claim 19, wherein the adjusting comprises normalizing a frequency response of the first input source.

22. The method of claim 16, further comprising:
outputting an audible signal representative of a selected phoneme to an external speaker, the first input received at the first input source and the ambient sound received at the second input source comprising the audible signal;
displaying, on a display, one or more phonemes including the selected phoneme; and
adjusting one of the first input source, the stimulator, the first signal processor, or a transfer function in response to a user input selecting the one or more phonemes.

23. The method of claim 16, wherein the first input source is part of the cochlear implant system and the second input source is part of the external device.

24. The method of claim 16, further comprising outputting the stimulation signal and the second input signal to a second external device.

25. The method of claim 24, wherein the second external device is configured to:
compare at least two of the stimulation signal, the first input signal, or the second input signal;
determine if correction is required based on the comparison; and
if correction is required, updating operation of at least one of the first input source, the stimulator, the first signal processor, or a transfer function programmed into the first signal processor.

26. The method of claim 16, wherein the external device comprises the memory.

27. A method of adjusting a cochlear implant system comprising:
pairing a cochlear implant system with an external device comprising a speaker, the cochlear implant system comprising an input source configured to generate an input signal representative of ambient sound and a signal processor programmed to output a stimulation signal to a stimulator based in part on the input signal;
receiving a selection of a phoneme, word, or phrase via the external device;
outputting the selection of the phoneme, word, or phrase to the cochlear implant system via the speaker;
receiving an indication of an accuracy of the phoneme, word, or phrase via the external device; and
adjusting at least one of the input source, the signal processor, the stimulator, or a programmed transfer function of the signal processor, based on the received indication of the accuracy of the phoneme, word, or phrase.
